(19) United States
(12) Patent Application Publication
(10) Pub. No.: US 2026/0114936 A1
(43) Pub. Date: Apr. 30, 2026

(54) SYSTEM AND METHOD FOR REAL-TIME SURGICAL VISUALIZATION USING AUGMENTED REALITY

(71) Applicant: MedAlign AR Inc, Westmount (CA)

(72) Inventors: Ahmed Aoude, Beauharnois (CA); Alireza Madani, Montreal (CA); Robert Turcotte, Laval (CA); Seyed Amir Hooshiar Ahmedi, Brossard (CA)

(73) Assignee: MedAlign AR Inc, Westmount (CA)

(21) Appl. No.: 19/373,681

(22) Filed: Oct. 29, 2025

Related U.S. Application Data
(60) Provisional application No. 63/713,674, filed on Oct. 30, 2024.

(51) Int. Cl. A61B 34/20; A61B 90/00; G06F 3/01

(52) U.S. Cl. CPC A61B 34/20; A61B 90/37; G06F 3/013; A61B 2034/2055; A61B 2090/365; A61B 2090/372

(57) ABSTRACT

A system and method for decentralized augmented reality assisted stereotaxic surgical guidance including a surgical navigation unit having motion tracking unit, an augmented reality head mounted display (AR-HMD) providing an augmented reality camera scene (AR-scene), and at least one tracked object. The AR-HMD and tracked object(s) include tracking marker arrays located thereon. A processing unit is provided in wireless communication with the AR-HMD for processing tracking data from the surgical navigation unit in conjunction with data from AR-HMD to enable an accurate, real-time mixed reality scene displayed to a wearer of the AR-HMD.

### TECHNICAL FIELD

The invention generally relates to surgical methods and apparatus. More particularly, the present invention relates to computer-assisted bone surgery including methods and apparatus for planning, guidance, and autonomous execution therefor.

### BACKGROUND OF THE ART

Surgical navigation systems aid surgeons during interventions by tracking medical tools in relation to the patient's anatomy. With conventional systems, however, surgeons have to divide their attention between navigation information on a 2D monitor and the patient. With the advent of augmented reality head mounted displays (AR-HMD), also referred to as AR goggles, navigation information may be displayed within the surgeon's view of the operating site. Thus, the surgeon can therefore focus on the patient only. Compared to conventional navigation systems, AR-HMDs are inexpensive and have a slim form factor. This allows implementation outside of the operating room opening up new areas of application.

A variety of tracking mechanisms are known in the context of AR-HMDs. Inside-out tracking is a method of tracking that uses cameras built into the frontal portion of an AR-HMD. However, such known methods of tracking have limited accuracy which makes them unsuitable for surgical applications needing precision. As well, known image markers are sensitive to occlusions and different light conditions, and they must constantly face the cameras for tracking. In practice, this limits the trackable movements of objects. One known attempt at applying AR-HMDs to surgical applications is discussed in “Inside-Out Instrument Tracking for Surgical Navigation in Augmented Reality” by Gsaxner et al, VRST '21: Proceedings of the 27th ACM Symposium on Virtual Reality Software and Technology, Article No.: 4, Pages 1-11, https://doi.org/10.1145/3489849.3489863, published Dec. 8, 2021 and herein incorporated by reference.

While AR-HMDs are capable of projecting virtual scene objects onto the visor through which the user views the surrounding world as discussed above, there are always serious challenges in design of AR devices because all of them must perform numerous computationally-expensive background software threads which are continuously scanning the surroundings and localizing the device inside the room to be able to properly anchor virtual objects in the AR scene. Although the spatial localization and perception of such devices are currently being realized using time-of-flight (ToF) depth cameras typically having four visible light cameras, two eye-tracking infrared sensors, and three inertial measurement unit (IMU) sensors, the real-time fusion and data processing of these nine sensing modalities is computationally heavy and still not accurate enough to be used for surgical applications. The goal in AR-assisted surgical applications is to be able to visualize the 3D models of anatomy inside the AR interface in a way that it matches the reality.

Due to the limited computational capability of AR systems with favorable refresh rates and considerable energy consumption of such real-time processes, localization of navigated scene objects in a surgical scenario (e.g., navigated anatomy through a tracking marker array (TMA), navigated surgical instruments, or even navigated AR goggles) cannot be relegated to the conventional on-board processing within an AR-HMD. Moreover, with the advent of AR goggles throughout the past decade in almost all domains, noticeably commercial and entertainment and, most recently, medicine and surgery, the demand for robust and accurate projection of 3D objects in the AR scene has constantly been rising. Particularly for computer-assisted spine and musculoskeletal (MSK) surgeries, AR projection accuracy and stability in rendering actual anatomical 3D models play an essential role in gaining a surgeon's trust in using AR technologies.

What is therefore needed is a robust surgical navigation system and related method to alleviate the problems of applying AR technology to surgery applications.

### SUMMARY

The present disclosure provides a robust surgical AR navigation system and related method and apparatus to alleviate the problems of applying AR technology to surgery applications. Within the context of rigid anatomy surgery, the present disclosure provides for computer-assisted preoperative planning, intraoperative guidance, and autonomous surgery-on-bones methods.

The present disclosure also provides localization of navigated scene objects in a surgical scenario such as, but not limited to, navigated anatomy through a TMA, navigated surgical instruments, or navigated AR-HMDs whereby localization is delegated to the hardware dedicated to solely the object tracking task which is the surgical navigation system.

The present disclosure also provides a system and method for decentralized augmented reality assisted stereotaxic surgical guidance including a surgical navigation unit having a motion tracking unit, an augmented reality head mounted display (AR-HMD) providing an augmented reality camera scene (AR-scene), and at least one tracked object. The AR-HMD and tracked object(s) include tracking marker arrays located thereon. A processing unit is provided in wireless communication with the AR-HMD for processing tracking data from the surgical navigation unit in conjunction with data from AR-HMD to enable an accurate, real-time mixed reality scene displayed to a wearer of the AR-HMD.

The present disclosure provides a system for augmented reality assisted stereotaxic guidance, the system comprising: a surgical navigation unit having a motion tracking unit; an augmented reality head mounted display (AR-HMD) providing an augmented reality camera scene (AR-scene), the AR-HMD including a first tracking marker array located thereon; at least one tracked object including a second tracking marker array located thereon; and a processing unit located separately from, and operably connected to, the surgical navigation unit for receiving preprocessed data representing tracking data correlated to the tracking marker arrays; wherein the processing unit is wirelessly connected to the AR-HMD communicating the AR-scene data thereto and the processing unit provides processed data to the AR-HMD thereby enabling a mixed reality scene displayed to a wearer of the AR-HMD.

The present disclosure also provides: that the processed data is provided to the AR-HMD in real-time; that the at least one tracked object is a portion of a patient anatomy; that the portion of the patient anatomy is a human bone element; that the processing unit is in communication with the surgical navigation unit; that the processmg unit is wirelessly connected to the AR-HMD via a private WiFi network; that the processing unit co-registers the AR-scene with the surgical navigation unit; that the processing unit provides co-registration by synchronous data acquisition and recording through both the surgical navigation unit and the AR-HMD whereby a data frame containing concatenated synchronously stacked data is received by the processing unit, and that the navigation unit reports in each time instant for each of the tracking marker arrays to the processing unit an array in accordance with the following equation

$$pos\_quat ( t ) = [ x ( t ) , y ( t ) , z ( t ) , qw ( t ) , qx ( t ) , qy ( t ) , qz ( t ) ]$$

defining a cartesian position of a coordinate system of each TMA and a quaternion representation of an orientation of each TMA frame with respect to a reference frame of the surgical navigation unit.

The present disclosure also provides a method for augmented reality assisted stereotaxic guidance for an object using the system as mentioned above, the method comprising: obtaining an optical frame for an augmented reality head mounted display (AR-HMD) having a first tracking marker array located thereon; computing a co-registration matrix based data streams from the AR-HMD and a surgical navigation unit having a motion tracking unit; calibrating, in real-time, a correction matrix thereby enabling a mixed reality scene displayed to a wearer of the AR-HMD which matches reality.

The present disclosure also provides: that the calibrating compensates for both eye anatomy and distance to the object of the wearer of the AR-HMD; that the optical frame is based upon the wearer's binocular pupillary distance and eye focal distance, and that the optical frame is produced internal to the AR-HMD using one or more eye-tracking camera.

### BRIEF DESCRIPTION OF THE DRAWINGS

Having thus generally described the nature of the invention, reference will now be made to the accompanying drawings, showing by way of illustration example embodiments thereof and in which:

FIG. 1A is a schematic showing a decentralized system architecture showing connections between blocks to indicate data flow in accordance with the present invention.

FIG. 1 is a schematic showing components of the AR scene consisting of the surgical navigation system, AR goggle, anatomy and their respective IR marker tracking marker arrays in accordance with the present invention.

FIG. 2 is a depiction of a sample test-bench which is used for developing the time-variant eye-correction matrix in accordance with the present invention.

FIG. 3 depicts the deviation of observed and template patterns with exaggeration for visualization and demonstration purposes in accordance with the present invention.

FIG. 4a shows the operator probing the two sets of template and observed points while being in the close-up head operating distance in accordance with the present invention.

FIG. 4b shows the operator performing the same scans on a farther head distance in accordance with the present invention.

FIGS. 4c and 4d, respectively, depict the close-up and further out surgeon positions with regard to the AR-scene after the eye-correction matrix real-time adaptation parameters are computed and it is compensating the for the deviations in real-time in accordance with the present invention.

FIG. 5 Constitution of the two coordinate systems which are being used to compute the eye-correction fine-tuned calibration matrix.

FIGS. 6a and 6b illustrate graphically two possible modes for the adaptation function in accordance with the present invention.

### DETAILED DESCRIPTION

The present disclosure will be discussed in terms of first a system and secondly a methodology used in conjunction with the system. The system relates to computationally decentralized AR-assisted stereotaxic guidance as an overall architecture utilizing surgical navigation systems and AR-goggles. Such navigation systems and AR-goggles are known and will not be further described herein in detail. The methodology relates to a real-time, fiducial-free, and adaptive framework for registering AR projectors in navigation systems. It should be understood that the system and methodology involve AR-assisted stereotaxic surgical procedures such as, but not limited to, orthopedic, spinal, cranial, or maxillofacial whereby several scene elements (e.g., the AR-goggles worn by the surgeon and the object being tracked) are being individually tracked by the surgical navigation system.

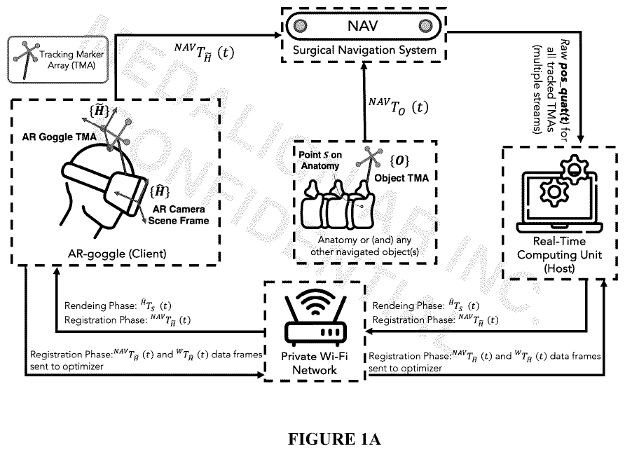

All components in the AR-assisted surgery such as AR-goggle, target anatomy and navigated surgical tools, each have their own TMA connected rigidly to their structure. To be able to reach the goal of AR-assisted surgery which is matching the rendered 3D models of the anatomy inside the AR-scene to the real-world's actual object it is required to co-register the navigated AR-goggle's scene with the surgical navigation system. Specific techniques to register the tracked anatomy into the navigated AR-scene are further discussed later hereinbelow with regard to the present methodology. Here, the system providing computationally decentralized AR-assisted stereotaxic guidance will be discussed as shown in FIG. 1A.

In accordance with an embodiment of the present invention, FIG. 1A represents the computationally decentralized AR-assisted stereotaxic surgical system. To be able to run the inventive methodology on the AR-goggle computationally as cost-effectively as possible and keep the communication untethered, the depicted workflow has been developed. As shown, the system includes an AR-goggle with a corresponding TMA and an object such as a point on the tracked object (i.e., patient anatomy) with a corresponding TMA. The AR-goggle may be any known type of augmented reality headsets such as, but not limited to, Google Glass Enterprise 2, Microsoft HoloLens 2, Lenovo ThinkReality A3, Apple Vision Pro, Vuzix Blade Upgraded, or the like. The TMAs of the AR-goggle and navigated object are optically tracked by a surgical navigation system (NAV) while the surgical navigation system is in wired or wireless communication with a real time computing unit. The AR-goggles and real time computing unit are wirelessly connected.

The surgical navigation system tracks multiple TMAs in real-time. In the scenario where the goal is to render a point S on the point cloud of a tracked anatomy with TMA {O} inside the AR-goggles scene frame {H}, there are several steps that should be taken. These include: co-registration; object and AR-goggle tracking; and anatomy localization and rendering.

In terms of co-registration, this step requires synchronous data acquisition and recording through both the navigation and AR-goggle spatial perception engines to solve for the co-registration problem (finding

$${}_{}^{\tilde{H}} {T}_{\hat{H}}^{} ) .$$

In this phase, the navigation system tracks the TMA attached to the AR-goggle and broadcasts its position data

$$( {}_{}^{NAV} {T}_{\tilde{H}}^{} ( t ) ( t ) ) .$$

Inis position data will be synchronously recorded alongside the position of the AR-camera frame ({Ĥ}) with respect to the AR-goggle spatial perception engine's world reference ({W}) frame. A data frame containing concatenated synchronously stacked data will be sent back to the real-time computing unit for solving the calibration problem. Details of the co-registration step are further discussed later hereinbelow with regard to the present methodology. It should be understood that this two-way communication is accomplished wirelessly though Internet protocols such as, but not limited to, Transmission Control Protocol (TCP) via a suitable private Wi-Fi network as shown.

With regard to the step of object and AR-goggle tracking, again a TMA is provided on the AR-goggle and tracked object. It should be understood that the object may be the given patient anatomy itself and/or any other navigated object including, but not limited to, surgical equipment. In each time instant, position of each TMA in the scene is tracked by the surgical navigation system (NAV) and sent to the real-time computing unit (Host). The computing unit may be any suitable processor device known in the computing art including, but not limited to, a personal computer, laptop, tablet, remote server including, but not limited to cloud computing, or the like. As well, processing may include a component of such that may be remote from the surgical arena such as, but not limited to, cloud-based processing. Note that in FIG. 1A,

$${}_{}^{NAV} {T}_{\hat{H}}^{} ( t )$$

which represents the position of the AR-goggle TMA with respect to the navigation system's local reference frame and

$${}_{}^{NAV} {T}_{O}^{} ( t )$$

which represents the position of the target navigated object TMA with respect to the navigation system's local frame are not actual homogenous transformation matrices. The values which the navigation system reports in each time instant for each TMA to the computing unit is a 7×1 array in accordance with the following equation:

$$pos\_quat ( t ) = [ x ( t ) , y ( t ) , z ( t ) , {q}_{w} ( t ) , {q}_{x} ( t ) , {q}_{y} ( t ) , {q}_{z} ( t ) ] \qquad Equation 1$$

Equation 1 indicates the cartesian position of the coordinate system of each TMA as well as the quaternion representation of the orientation of each TMA frame with respect to the frame defined as the reference of navigation systems' motion tracking camera. In terms of the surgical navigation system itself including the tracking camera, it should be understood and readily apparent to one of skill in the art that such navigation and tracking devices are known in the art and will not be further described herein and may include any suitable modality of tracking including, but not limited to, infrared, optical, electromagnetic, or the like.

The registration matrix is solved using hand-eye coordination solutions. The matrix manipulation and multiplication processes are done on the real-time computing unit with a fast update rate. The quick update rate is facilitated because only the computed position and orientation of point S with respect to AR-camera frame {Ĥ} is being broadcasted in real-time to the software client on the AR-goggle. By sending only the already-computed, real-time

$${}_{}^{\hat{H}} {T}_{S}^{} ( t )$$

to the AR-goggle's onboard software for visualization, the computational load is considerably lessened from the on-board hardware and the system remains untethered and user-friendly (i.e., wireless and easily worn by a surgeon) while maintaining the optimal accuracy levels as accurate as those provided by medical-standard surgical navigation systems. Therefore, continuous AR viewing in real-time of the given tracked anatomy is accomplished with high levels of accuracy that is necessary and required in surgical settings.

As the overall system architecture has been generally described above, the underlying methodology in accordance with the present invention will now be described in detail. The inventive methodology provides a framework which incorporates the robustness and accuracy of surgical navigation systems into the AR rendering capabilities of spatial-aware AR devices. To do so, first, the following description distills the complex and labyrinthine problem of AR goggle-NAV co-registration into a hand-eye calibration problem. Second, the following description introduces a fiducial-free approach to determining the hand-eye calibration matrix using an AR goggle's spatial localization capabilities. Third, the following description provides a fine-tuning method which adaptively accounts for both 1) each user's eyeball anatomy-induced AR scene distortions (i.e., paralex effects) and 2) eye distance from the target scene in real-time. Advantageously, the following discussion shows that the present invention provides projection of graphical scene landmarks with precision accuracy into the physical real-world through the AR goggle. Thus, the present invention plays a critical role in the general use of AR goggles alongside surgical navigation systems for spine and MSK surgery applications.

In this section, we will first fundamentally constitute the problem of co-registering the surgical navigation system (NAV) and spatial-aware augmented reality (AR) goggles by defining multiple cartesian coordinate frames involved in our computations. Secondly, we present a fiducial-free hand-eye calibration method to solve for the main NAV-AR co-registration matrix. Third, we present an adaptive fine-tuning re-calibration method which accounts for each user's intrinsic eye-induced AR projection distortions and head distance to the target real-world scene. Before delving into mathematical modeling, we shed light into the preferred outcome.

It should be noted that, in accordance with the present disclosure, the term “spatial-aware AR goggle” mentioned above refers to AR googles such as Microsoft Hololens 2 or Apple Vision Pro which have the capability of locating their mixed reality (MR) scene camera coordinate system in real-time inside the room where the goggle is located. This has become possible by incorporating sensor fusion techniques to use the data from depth (ToF) sensors, IMU (inertial measurement sensors such as accelero-magentometers and gyroscopes), and head tracking cameras to accurately locate the position and orientation of the MR camera frame with respect to an anchored-world-reference frame which is arbitrarily defined and anchored statically in the room in which the AR goggle is located at the initialization phase of the implemented AR software.

It is a desirable outcome of the present disclosure to be able to render scene objects representing physical landmarks that are rigidly located with respect to a reference NAV tracking array in the real-world (physical) environment into the AR camera mixed-reality scene (hereinafter referred to as the “MR scene”). The intent is to render these landmarks through the AR goggle in a way that it matches the actual location of these landmarks in the real-world.

In this manner, one may easily have the coordinates of each point of the point cloud of a 3D model of a target anatomy (e.g., a vertebral body having a NAV tracking array rigidly connected to the spinous process) and be able to render the 3D model of the anatomy through the AR goggle while each point on the target anatomy in the physical world matches the 3D rendered MR scene object. It should be noted that the 3D model of a rigid anatomy rigidly connected to a NAV tracking array may be scanned and extracted with available technologies such as O-Arm from Medtronic Inc. This provides a key enabling technology in a multitude of rigid anatomy surgical interventions such as reconstruction (including, but not limited to arthoplasty) for knee, hip, and shoulder joints, spine, head and neck, craniomaxillofacial and otorhinolaryngology and basically any intervention which allows a NAV tracking array to be rigidly connected to a rigid anatomy.

In terms of the problem solved by the present disclosure, it should be noted that there have always been concerns raised by surgeons about inadvertently bumping and displacing the NAV tracking array which is supposed to be rigidly connected to the bony anatomy. This is because after one O-Arm scan is done the coordinates of each point of the anatomy point cloud relative to the NAV tracking array is no longer valid. Hence, throughout this disclosure the rigid-body contact assumption for navigation array marker and solid anatomy remains valid.

FIG. 1 schematically depicts the principal elements of the proposed framework. To render any point S on the three-dimensional model of the anatomy—rigidly connected to a tracking marker array (TMA)—exactly at its true physical location within the mixed-reality (MR) scene, the system leverages the proven accuracy of the navigation (NAV) system to localize point S in the MR environment. Achieving this requires continuous determination of the unknown headset coordinate frame {Ĥ:} in real time while simultaneously tracking the navigated anatomy's TMA {O}. By jointly navigating {Ĥ:} and {O} and utilizing the real-time 3D rendering capabilities of a graphical engine (e.g., Unity3D), the invention allows continuous visualization of any point S on the anatomy without large incisions or exposure. The optical frame {Ĥ:} of the AR camera is positioned according to the user's binocular pupillary distance and focal length, computed by the goggle's internal eye-tracking system during the initial user-specific calibration procedure. To localize {Ĥ:} with NAV, a TMA is rigidly affixed to the AR goggle as shown in FIG. 1. Tracking {H} and {O} in real time permits computation of the position of any point S with respect to {Ĥ:} as follows:

$${}_{}^{\hat{H}} {T}_{S}^{} = {}_{}^{\hat{H}} {T}_{O}^{} {}_{}^{O} {T}_{S}^{} \qquad Equation ( 1 a )$$

where \hat{ }O T_S is known from pre-acquired medical imaging. Without loss of generality, S may be represented by its own coordinate system {S}. The key to rendering point S in the MR scene therefore reduces to determining the constant homogeneous transformation matrix \hat{ }H T_H.

The AR-NAV co-registration problem is thus reduced to finding the homogeneous transformation matrix \hat{ }H T_H, which remains constant over time since {Ĥ:} and {H} are rigidly fixed relative to one another. Spatial-aware AR goggles compute the position of {Ĥ:} relative to an anchored world reference {W} established at software initialization. As the navigation system {NAV} is stationary during calibration, {NAV} and {W} are fixed with respect to each other. Consequently, both \hat{ }{NAV} T_H and \hat{ }W T_H remain constant even as the headset moves. Fusion of the AR goggle's peripheral-perception data with the NAV-streamed TMA position enables a fiducial-free computation of the co-registration matrix using a dual-unknown hand-eye calibration formulation. The relative transform between {W} and {NAV} is expressed as:

$${}_{}^{\dot{H}} {T}_{O}^{} = {( {}_{}^{NAV} {T}_{\tilde{H}}^{} {}_{}^{\tilde{H}} {T}_{\hat{H}}^{} )}^{- 1} {}_{}^{NAV} {T}_{O :} \qquad Equation ( 2 )$$

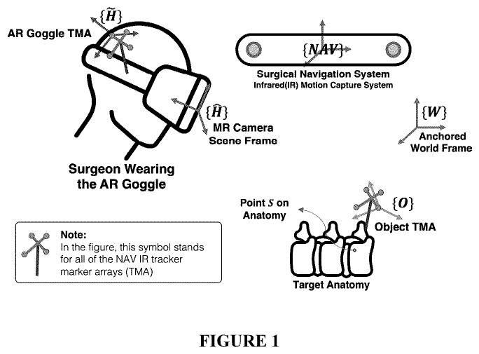

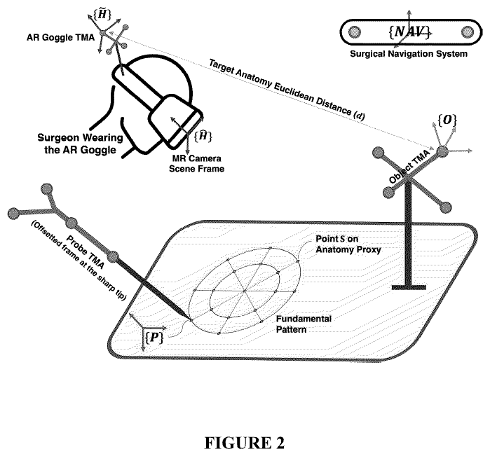

which can be reformulated as a classical AX=YB problem:

$${A}_{( i )} X = {YB}_{( i )} , i ∈ \{ 1 , 2 , 3 , … , {n}_{s} \} \qquad Equation ( 3 )$$

where each time-sampled pair corresponds to synchronized position data from both systems. Solving this yields the time-invariant matrices \hat{ }H T_W and \hat{ }{NAV} T_W, from which the AR-NAV co-registration transform \hat{ }H T_H is derived. Once computed, the co-registration remains valid for subsequent sessions, as user-specific deviations are later compensated by the adaptive re-calibration process. To validate the computed \hat{ }H T_H, a test bench (FIG. 2) is used. It comprises the surgical navigation system, a tracked AR headset worn by the user, and a rigid plate serving as an anatomy proxy with a TMA rigidly attached. The plate bears a printed pattern of seventeen reference points whose coordinates relative to {O} are determined by probing.

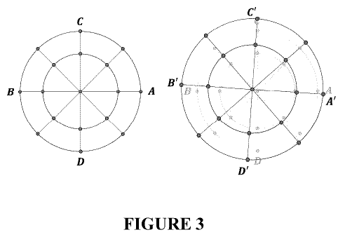

Although the co-registration permits correct projection of point S, minor distance- and optics-dependent deviations persist. FIG. 3 exaggerates these deviations for clarity. They vary with viewing distance and individual ocular parameters (e.g., corrective lenses or alignment). Thus, an adaptive eye-correction matrix is introduced to compensate these residual distortions in real time:

$${}_{}^{\hat{H}} {T}_{S}^{} = {}_{}^{\hat{H}} {T}_{O}^{} {}_{}^{O} {T}_{{S}^{′}}^{} {}_{}^{{S}^{′}} {T}_{S}^{} \qquad Equation ( 4 )$$

where \hat{ }{S′} T_S is the adaptive correction matrix accounting for user-specific and distance-related projection errors. This matrix continuously aligns the observed MR renderings with their true physical counterparts.

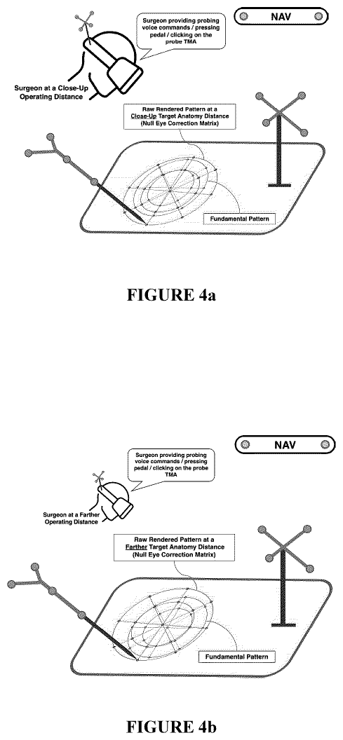

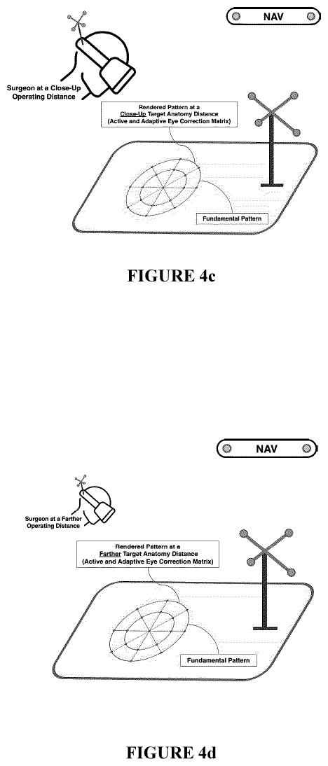

FIG. 4A shows a user probing four calibration landmarks (A, B, C, D) on the real plate, then the corresponding rendered points (A′, B′, C′, D′) in the MR scene, both at near and far working distances (FIGS. 4A-4B). The probe data define the template and observed coordinate systems, {Tem} and {Obs}, from which the correction transform is derived:

$${}_{}^{Obs} {T}_{Tem}^{} = {( {}_{}^{O} {T}_{Obs}^{} )}^{- 1} {}_{}^{O} {T}_{Tem}^{} \qquad Equation ( 5 )$$

Two such matrices—one for near (d_{c}) and one for far (d_{f})—are computed. Their respective rotation components are mapped to Euler-angle sets {α(c), β(c), γ(c)} and {α(f), β(f), γ(f)}, and translation components are extracted from the last column of each homogeneous matrix.

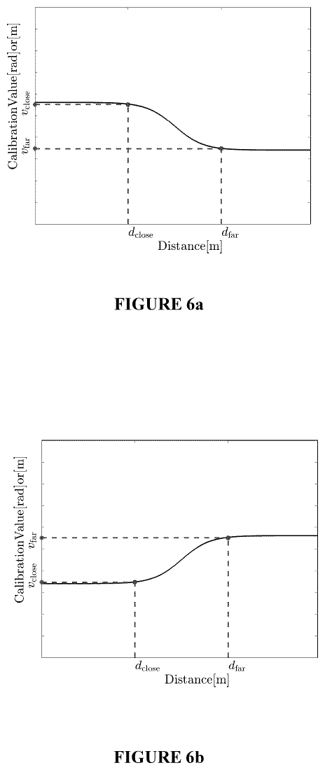

To enable smooth adaptation with changing head-object distance d, each parameter v(d) is continuously updated according to:

$$v ( d ) = \frac{{v}_{f} + {v}_{c}}{2} + \frac{{v}_{f} - {v}_{c}}{2} tanh ( Γ ( d - \frac{{d}_{c} + {d}_{f}}{2} ) ) \qquad Equation ( 6 )$$

where Γ is an empirically determined shaping coefficient. FIG. 6 illustrates the two representative adaptation profiles.

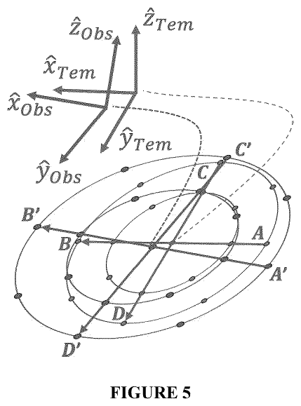

Stretch-based distortions are further compensated by computing scaling factors δ(c) and δ(f) for the near and far distances, updated in real time using the same functional model. FIG. 5 schematically presents the template and observed coordinate frames and their spatial relationships, while FIGS. 6A-6B illustrate the adaptation functions governing the continuous correction of rotational and positional deviations as the surgeon's head position changes.

1) A system for augmented reality assisted stereotaxic guidance, the system comprising:
a surgical navigation unit having a motion tracking unit;
an augmented reality head mounted display (AR-HMD) providing an augmented reality camera scene (AR-scene), the AR-HMD including a first tracking marker array located thereon;
at least one tracked object including a second tracking marker array located thereon; and
a processing unit located separately from, and operably connected to, the surgical navigation unit for receiving preprocessed data representing tracking data correlated to the tracking marker arrays;
wherein the processing unit is wirelessly connected to the AR-HMD communicating the AR-scene data thereto and the processing unit provides processed data to the AR-HMD thereby enabling a mixed reality scene displayed to a wearer of the AR-HMD.

2) The system as claimed in claim 1, wherein the processed data is provided to the AR-HMD in real-time.

3) The system as claimed in any one of claims 1 to 2, wherein the at least one tracked object is a portion of a patient anatomy.

4) The system as claimed in any one of claim 1 to claim 3, wherein the portion of the patient anatomy is a human bone element.

5) The system as claimed in any one of claim 1 to claim 4, wherein the processing unit is in wired or wireless communication with the surgical navigation unit.

6) The system as claimed in any one of claim 1 to claim 5, wherein the processing unit is wirelessly connected to the AR-HMD via a private WiFi network.

7) The system as claimed in any one of claim 1 to claim 6, wherein the processing unit co-registers the AR-scene with the surgical navigation unit.

8) The system as claimed in claim 7, wherein the processmg unit provides co-registration by synchronous data acquisition and recording through both the surgical navigation unit and the AR-HMD whereby a data frame containing concatenated synchronously stacked data is received by the processing unit.

9) The system as claimed in claim 8, wherein the navigation unit reports in each time instant for each of the tracking marker arrays to the processing unit an array in accordance with the following equation
$$pos\_quat ( t ) = [ x ( t ) , y ( t ) , z ( t ) , qw ( t ) , qx ( t ) , qy ( t ) , qz ( t ) ]$$
defining a cartesian position of a coordinate system of each TMA and a quaternion representation of an orientation of each TMA frame with respect to a reference frame of the IR mechanism of the surgical navigation unit.

10) A method for augmented reality assisted stereotaxic guidance for an object using the system as claimed in any one of claims 1 to 9, the method comprising:
obtaining an optical frame for an augmented reality head mounted display (AR-HMD) having a first tracking marker array located thereon;
computing a co-registration matrix based data streams from the AR-HMD and a surgical navigation unit having a motion tracking unit;
calibrating, in real-time, a correction matrix thereby enabling a mixed reality scene displayed to a wearer of the AR-HMD.

11) The method as claimed in claim 10, wherein calibrating compensates for both eye anatomy and distance to the object of the wearer of the AR-HMD.

12) The method as claimed in any one of claims 10 to 11, wherein the optical frame is based upon the wearer's binocular pupillary distance and eye focal distance.

13) The method as claimed in claim 12, wherein the optical frame is produced internal to the AR-HMD using one or more eye-tracking camera.

14) A system and method for decentralized augmented reality assisted stereotaxic surgical guidance as show in the drawings and described in the disclosure.
